(19) United States
(12) Patent Application Publication
(10) Pub. No.: US 2026/0236939 A1
(43) Pub. Date: Aug. 13, 2026

(54) ARTIFICIAL INTELLIGENCE SYSTEM FOR REAL-TIME, ACCURATE AND TARGETED BUSINESS-TO-BUSINESS INFORMATION

(71) Applicants: Osama Naim Abdelhadi, Dubai (AE); Oday Ali Alattal, Dubai (AE)

(72) Inventors: Osama Naim Abdelhadi, Dubai (AE); Oday Ali Alattal, Dubai (AE)

(21) Appl. No.: 19/455,053

(22) Filed: Jan. 21, 2026

Related U.S. Application Data
(60) Provisional application No. 63/756,973, filed on Feb. 11, 2025.

(51) Int. Cl. G06Q 30/018; G06Q 10/107

(52) U.S. Cl. CPC G06Q 30/018; G06Q 10/107

(57) ABSTRACT

A real-time, AI-powered B2B data sourcing and verification system with unparalleled accuracy, niche targeting capabilities, and live updates, surpassing traditional, static database solutions.

### CROSS REFERENCE TO RELATED APPLICATION

This application claims the benefit of priority of U.S. provisional application No. 63/756,973, filed Feb. 11, 2025, the contents of which are herein incorporated by reference.

### BACKGROUND OF THE SUBJECT DISCLOSURE

The present invention relates to business-to-business (B2B) systems, and more particularly, to an AI-powered system for real-time, accurate, enriched, and niche-targeted B2B contact databases.

Current B2B data solutions rely on static databases, which must be updated in batches, leading to stale information that can be inaccurate, or incomplete. Additionally, B2B data solutions rely on conventional data sources and verification methods which fail to capture niche-specific data or verify contact information with high precision because they source data on an “as-is” basis and use limited verification techniques. Furthermore, data crawling methods associated with B2B data solutions provide limited data volume capabilities, resulting in missed opportunities within the Total Addressable Market (TAM). As such businesses struggle to obtain real-time, niche-specific, and high-accuracy contact details critical for effective marketing, sales, and outreach efforts.

As can be seen, there is a need for a real-time, AI-powered B2B data sourcing and verification system with unparalleled accuracy, niche targeting capabilities, and live updates, surpassing traditional, static database solutions.

### SUMMARY OF THE SUBJECT DISCLOSURE

In one aspect of the present subject disclosure, a real-time artificial intelligence system for business-to-business contact information management, including the following: a data sourcing module configured to query a plurality of heterogeneous data sources and collect raw contact data, the data sourcing module comprising: a data crawling framework configured to extract data from online sources; a contributor network integration module configured to receive data from contributor networks; and a plurality of partnership API connectors configured to interface with partner data sources; a data compliance module configured to perform compliance checks on the raw contact data against regulatory requirements including GDPR, CCPA, and PDPL regulations, wherein the data compliance module is configured to block non-compliant data and allow compliant data to proceed for further processing; an artificial intelligence matching algorithm module configured to align the compliant data with data enrichment requirements, the artificial intelligence matching algorithm module providing: a data footprint tracking module configured to track digital footprints of sourced data; and an email intelligence generation module configured to generate email intelligence; a data enrichment system configured to create unified enriched profiles by combining multiple data points from the artificial intelligence matching algorithm module, the data enrichment system including: a unified profile module configured to consolidate data points into unified profiles; and a dynamic update mechanism configured to continuously monitor for new data and automatically update the unified enriched profiles in real-time when new data becomes available; a catch-all verification system configured to verify email addresses in the unified enriched profiles, the catch-all verification system having the following: an email verification module configured to perform syntax validation, domain verification, and mail server response analysis; and a multilayered analysis module configured to perform additional verification; a phone number verification system configured to verify and enrich phone numbers in the unified enriched profiles, the phone number verification system including: a caller ID matching module configured to perform initial phone number validation; a geolocation engine configured to prioritize regionally relevant contacts; and a cloud communication AI-validation module configured to perform final phone number verification; a real-time search engine configured to provide live data retrieval from the unified enriched profiles, the real-time search engine including: a cloud infrastructure configured to ensure scalability; and a user interface dashboard configured to provide user interaction; a niche targeting artificial intelligence module configured to dynamically refine targeting based on user-defined criteria, the niche targeting artificial intelligence module having a behavioral analytics engine configured to identify nuanced behavioral patterns; and a niche-specific targeting engine configured to apply niche-specific targeting based on operational cues and user-defined prompts; one or more machine learning models configured to continuously improve system components through feedback learning, the one or more machine learning models including: at least one continuous learning model configured to learn from user interactions and data patterns; and at least one improvement pattern model configured to identify improvement patterns; and an API provisioning system configured to provide external access to the unified enriched profiles and enable integration with third-party applications, the API provisioning system further includes an integration layer module configured to facilitate third-party integration; and an access control module configured to control access to system resources.

In another aspect of the present subject disclosure, the real-time artificial intelligence system for business-to-business contact information management further includes wherein the data sourcing module is configured to iteratively query the plurality of heterogeneous data sources until requested data is found, wherein the catch-all verification system is configured to flag profiles with failed email verification for reprocessing by the data sourcing module and the artificial intelligence matching algorithm module to identify alternative email addresses, wherein the phone number verification system is configured to retry verification or employ alternative verification techniques when a phone number fails verification at any verification step, wherein the real-time search engine is configured to apply filters to search queries and check niche criteria, and wherein the real-time search engine is configured to refine and resubmit searches when no match is found, wherein the niche targeting artificial intelligence module is configured to trigger the behavioral analytics engine and the artificial intelligence matching algorithm module to dynamically identify matching profiles when user-defined niche criteria do not initially match existing profiles, wherein the one or more machine learning models are configured to: analyze user feedback to determine whether the user feedback is positive or negative; update the one or more machine learning models when the user feedback is positive; and adjust algorithms when the user feedback is negative, wherein the data compliance module is configured to control the API provisioning system, validate information from the real-time search engine, and monitor the data sourcing module, wherein the dynamic update mechanism is configured to: continuously monitor for new data availability;
trigger the data sourcing module to perform a dynamic update when new data is found; and enrich profiles with the new data, wherein the email verification module is configured to store verified email data and reprocess failed email data by querying additional data sources, wherein the phone number verification system is configured to store phone numbers as verified phone numbers only after the phone numbers pass caller IO matching verification, geolocation prioritization, and cloud communication AI verification, wherein the niche targeting artificial intelligence module is configured to identify: angel investors not explicitly listing their role but engaging in investment-related activities; eCommerce retailers by analyzing operational cues including add-to-cart features; and profiles based on user-defined prompts that differ from broader category classifications, wherein the system is configured to provide email accuracy of at least 98% and phone number accuracy of at least 95%, wherein the system provides access to over one billion contacts, wherein the unified enriched profiles include real-time updates of contact details including emails, phone numbers, and job titles, wherein the API provisioning system is configured to integrate with customer relationship management systems including Salesforce and HubSpot to automate lead generation, contact updates, and data retrieval workflows, wherein the behavioral analytics engine is configured to refine targeting based on industry-specific attributes and user-defined criteria, wherein the data sourcing module is configured to continue querying alternative data sources until data is located or all available sources are exhausted, wherein the system is configured to export data in formats selected from the group consisting of csv, Excel, PDF, and JSON, and wherein the niche targeting artificial intelligence module is configured to dynamically refine targeting in real-time based on user-defined prompts rather than relying on static lists.

These and other features, aspects and advantages of the present subject disclosure will become better understood with reference to the following drawings, description and claims.

### BRIEF DESCRIPTION OF THE DRAWINGS

FIG. 1 is a schematic diagram of an architecture of the system of an exemplary embodiment of the subject disclosure.

FIG. 2 is a flow diagram of a first plurality of methods of the system of an exemplary embodiment of the subject disclosure.

FIG. 3 is a flow diagram of a second plurality of methods of the system of an exemplary embodiment of the subject disclosure.

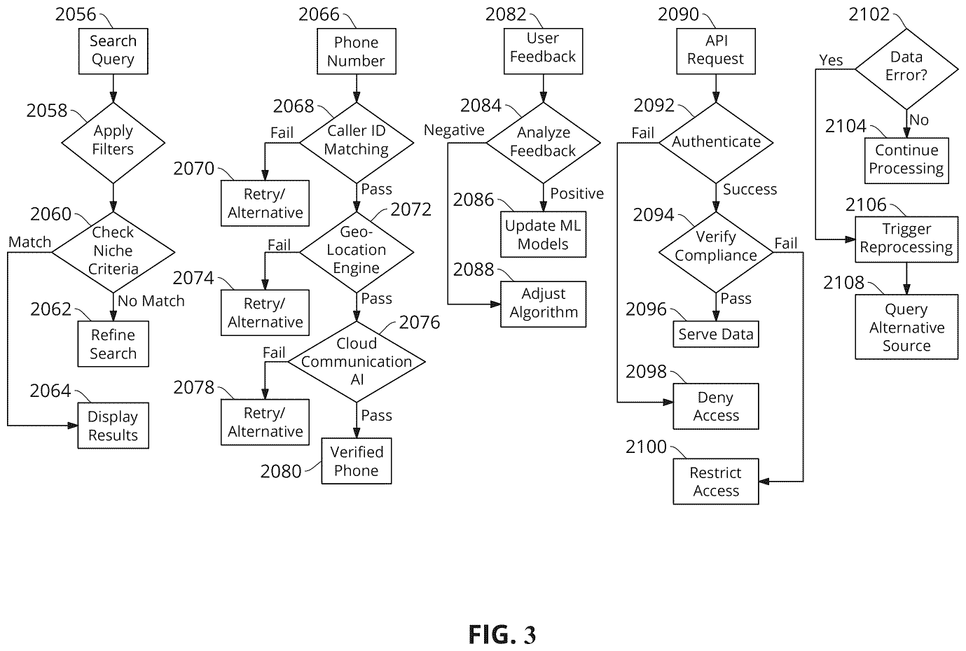

### DETAILED DESCRIPTION OF THE SUBJECT DISCLOSURE

The following detailed description is of the best currently contemplated modes of carrying out exemplary embodiments of the subject disclosure. The description is not to be taken in a limiting sense but is made merely for the purpose of illustrating the general principles of the subject disclosure, since the scope of the subject disclosure is best defined by the appended claims.

Broadly, an embodiment of the present invention provides a system including a dynamic, real-time database powered by AI and advanced algorithms, ensuring up-to-date, enriched, and highly accurate B2B contact information. The system of the present invention uses advanced AI algorithms to deliver continuously updated, enriched data with access to over one billion contacts, 98% email accuracy, 95% phone number accuracy, and the ability to target niche variables dynamically, addressing the limitations of static and conventional systems. Advantageously, the present invention uniquely enables niche-specific targeting and incorporates innovative methodologies to verify and enhance contact details, addressing the limitations of static databases and conventional practices.

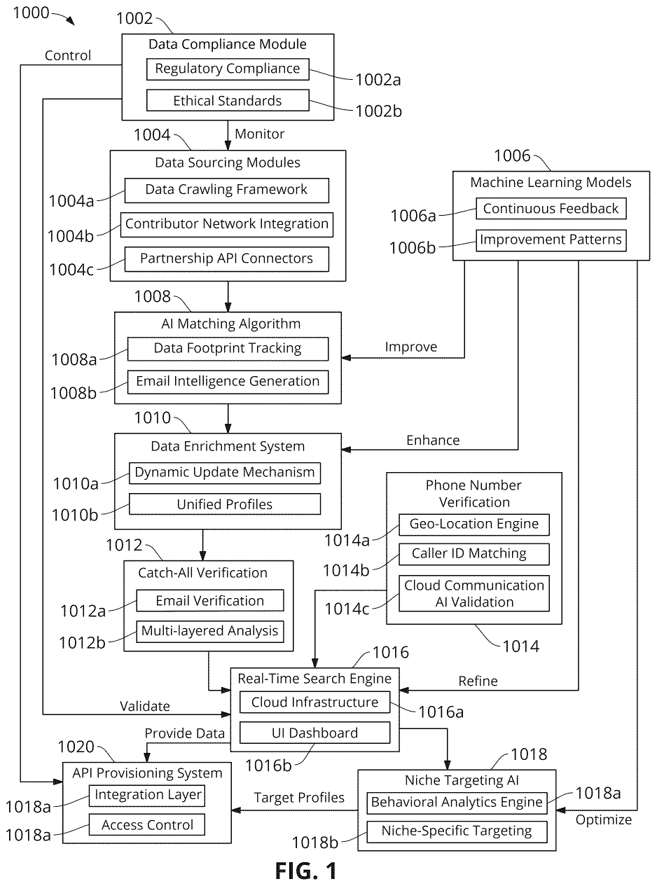

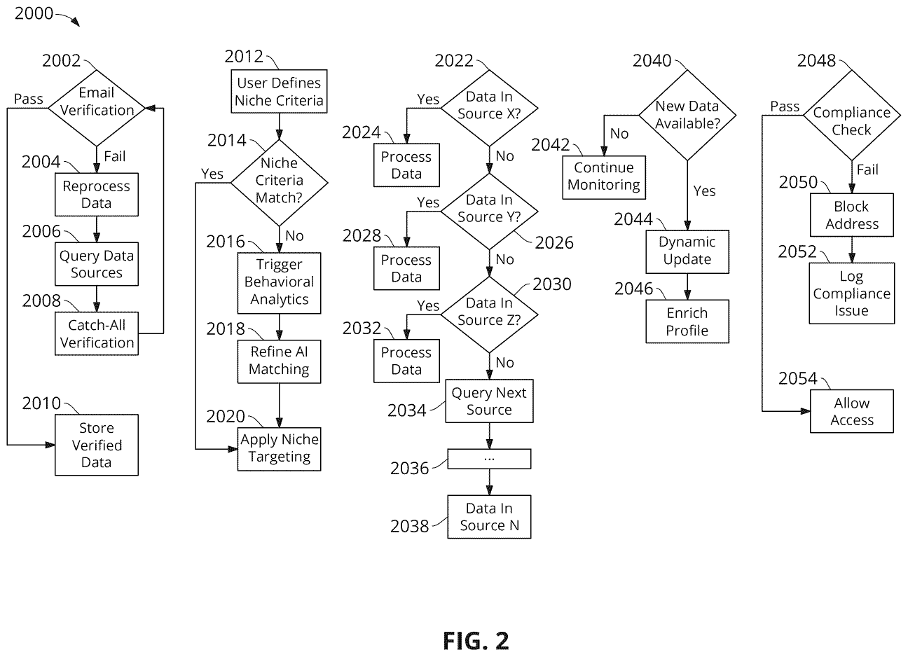

Referring now to FIGS. 1-2, aspects of the present invention are illustrated. FIG. 1 illustrates an architectural diagram of a system 1000 of the present invention. In embodiments, system 1000 can include a number of components, such as, but not limited to data compliance module 1002, data sourcing module 1004, machine learning models 1006, AI matching algorithms 1008, data enrichment system 1010, catch-all verification system 1012, phone number verification 1014, real-time search engine 1016, niche targeting AI 1018, and/or API provisioning system 1020. In embodiments, the plurality of components interact to provide the functionalities of system 1000.

Data compliance module 1002 can be configured to provide compliance to regulatory, legal, and ethical requirements. In embodiments, one or more sub-modules such as a regulatory compliance module and/or an ethical standards module 1002b can be configured to provide regulatory compliance and ethical compliance for system 1000. In embodiments, data compliance module 1002 can be configured to ensures all processes of system 1000, adhere to GDPR, CCPA, and PDPL regulations, while also maintaining legal and ethical compliance.

In embodiments, data compliance module 1002 can operate one or more compliance checks, as illustrated in FIG. 2. In embodiments, one or more data items can be submitted to compliance check 2048 and in the case the one or more data items passes the compliance check the one or more data items can be allowed access 2054 to one or more components of system 1000. In the case that the one or more data items fails the compliance check the one or more data items can be blocked 2050 and data compliance module 1002 can log a compliance issue. In embodiments, compliance check 2048 can include checks for compliance with GDPR, CCPA, and/or PDPL compliance. In embodiments, data compliance module 1002 can control access to system 1000 by controlling API provisioning system 1020, described further hereinafter, and can validate information from real-time search engine 1016, described further hereinafter, and finally, data compliance module 1002 can monitor data sourcing module 1004, described further hereinafter.

Data sourcing module 1004 can be configured to provide comprehensive and diverse data collection. In embodiments, one or more sub-modules can be provided to assist in data sourcing such as, but not limited to, a data crawling framework 1004a, a contributor network integration module 1004b, and/or one or more partnership API connectors 1004c. In embodiments, the one or more sub-modules can collect raw data from diverse, or heterogeneous, data sources for further processing by system 1000.

In embodiments, data sourcing module 1004 can operate one or more data collection operations, as illustrated in FIG. 2. In embodiments, data sourcing module 1004 can access a plurality of diverse data sources, wherein one or more queries can be issued for data from one or more data sources, wherein if the data is found in a data source it is processed (i.e. steps 2024, 2028, 2032, etc.). However, if data is not found in a data source, data sourcing module can issues a query to a next data source until the data is found (i.e. steps 2026, 2030, 2034, 2036, . . . , 2038). In embodiments, If new or updated data becomes available in the Data Sourcing Modules 1002, the Dynamic Update Mechanism 1010a automatically updates the corresponding enriched profile in real time to ensure accuracy and freshness.

AI matching algorithms 1008 can be configured to ensure data from data sourcing module 1004 aligns with requirements for data enrichment in system 1000. In embodiments, one or more sub-modules can be provided in AI matching algorithms 1008 such as, but not limited to, data footprint tracking modules 1008a and/or Email intelligence generation module 1008b. In embodiments, data footprint tracking module 1008a utilizing digital footprint tracking of data sourced to match and cross-verify data points.

One or more Machine Learning Models 1006 can be configured to continuously improve components, modules, or sub-modules of system 1000. In embodiments, the one or more machine learning models 1006 can include: at least one model for continuous learning 1006a which can learn from user interactions, data patterns, etc., to improve components of the system; and at least one improvement pattern model 1006b to improve components of system 1000. In embodiments, one or more machine learning models 1006 can improve the AI matching algorithms 1008 through feedback learning, and can enhance Data enrichment system 1010, described further hereinafter, through pattern improvement. Additionally, one or more machine learning models 1006 can refine real-time search engine 1016, described further hereinafter, and optimize Niche Targeting AI 1018, described further hereinafter.

In embodiments, machine learning models 1006 can perform one or more operations, as illustrated in FIG. 2. In embodiments, one or more user feedback can be provided to machine learning models 1006 at 2082. Analysis of the one or more user feedback can be performed at 2084 to determine if the feedback is positive or negative. In embodiments, if the one or more user feedback is positive the one or more machine learning models can be updated at 2086. Alternatively, if the one or more user feedback is negative, the one or more machine learning models can be adjusted.

Data Enrichment system 1010 can be configured to create unified and enriched profiles for system 1000. In embodiments, data enrichment system 1010 can include one or more sub-modules such as, but not limited to, dynamic update mechanism 1010a, and/or unified profile module 1010b. In embodiment, data enrichment system 1010, and/or one or more of the sub-modules can take processed data from the AI Matching Algorithm 1008 and combines multiple data points into a unified, enriched profile. In embodiments, dynamic update mechanism 1010a can ensure profiles are continuously updated to remain current.

In embodiments, dynamic update mechanism 1010a can operate one more dynamic update operations, as illustrated in FIG. 2. In embodiments, dynamic update mechanism 1010a can search, monitor, and/or crawl for new data 2040, in embodiments, monitoring can continue if no new data is found 2042. If new data is found, data sourcing module 1004 can perform a dynamic update 2044 of system 1000 by adding the newly found data. In embodiments, the newly found data can be enriched 2046.

Catch-all Verification 1012 can be configured to verify data points sourced and processed through system 1000. In embodiments, Catch-all verification 1012 can have one or more sub-modules such as, but not limited to an email verification module 1012a, and/or a multilayered analysis module 1012b. In embodiments email verification module 1012a can verify email accuracy from one or more enriched profiles by performing dynamic real-time checks such as syntax validation, domain verification, and mail server response analysis.

In embodiments, Catch-all verification can verify emails using one or more processes, illustrated in FIG. 2. In embodiments, one or more email data can be provided for verification at 2002, and in the case verification is passed the one or more email data can be stored in system 1000 at 2010. If the one or more email data fail verification they are reprocessed at 2004. In addition to reprocessing, one or more data sources can be queried for verification at 2006. Finally, catch-all verification can catch any email unverified using the above processes at 2008. In embodiments, If email verification fails, system 1000 can flag the profile for reprocessing and sends it to the Data Sourcing Modules 1004 and AI Matching Algorithm 1008 to identify alternative email addresses.

Phone number verification 1014 can be configured to verify and enrich phone numbers ingested into system 1000. In embodiments, phone number verification 1014 can include a plurality of submodules such as, but not limited to, a geolocation engine 1014a, a caller ID matching module 1014b, and/or, a cloud communication AI-validation module 1014c, to assist in verification and enrichment of phone numbers. In embodiments, phone verification module can validated phone numbers, and prioritize regionally relevant contacts to users of system 1000.

In embodiments, phone number verification 1014 using one or more sub-modules can perform one or more verifications, as illustrated in FIG. 2. In embodiments, one or more phone numbers can be ingested by system 1000 at 2066. The one or more phone numbers can be exposed to a first pass verification using caller ID matching at 2068. If the one or more phone numbers passes caller ID matching they can be provided to a geo-location engine at 272 for geographic prioritization. Additionally, once the one or more phone numbers passes geolocation prioritization they can be provided to cloud communication AI for verification at 2076. In embodiments, if the one or more phone numbers passes all verification steps they can be stored as verified phone numbers at 2080. However, if a failure is detected at any step the process may reattempt that step, or try alternative verification techniques (2070, 2074, and 2078).

Real-time Search Engine 1016 can be configured for live data retrieval and user interaction. In embodiments, real-time search engine 1016, through cloud infrastructure 1016a and/or UI dashboard 1016b, provide users with access to the enriched profiles via a live search capability and via UI dashboard 1016b. Advantageously, Cloud-Based Infrastructure 1016a ensures scalability, and the User Interface and Dashboard 1016b offer seamless user interaction.

In embodiments, real-time search engine 1016 can perform one or more operations, illustrated in FIG. 2. In embodiments, one or more search queries can be provided to real-time search engine at 2056. Additionally, one or more filters can be applied to the one or more search queries at 2058, and/or one or more niche criteria can be provided at step 2060. In the event of a match, search results can be displayed in real-time at 2064. In the event of no match search results can be refined and resubmitted at 2062.

Niche Targeting AI 1018 can be configured to leverages the enriched profiles and incorporate niche-specific targeting capabilities. In embodiments, niche targeting AI 1018 can include a plurality of sub-modules, such as, but not limited to a Behavioral Analytics Engine 1018a and a Niche-specific targeting engine 1018b. In embodiments, Behavioral Analytics Engine 1018a refines targeting based on industry-specific attributes or user-defined criteria. In embodiments, niche targeting AI 1018 though one or more of its sub-modules identifies nuanced behaviors like angel investors not explicitly listing their role but engaging in investment-related activities. Additionally, niche targeting AI 1018, can provide results based on operational cues (e.g., identifying eCommerce retailers by analyzing “add to cart” or other ecommerce features). Unlike static lists, the niche targeting AI 1018 dynamically refines targeting in real time based on user-defined prompts, such as finding “meal prep subscription companies” rather than broader “restaurant” category.

In embodiments, niche targeting AI 1018 can perform one or more operations, illustrated in FIG. 2. In embodiments, a user can define one or more niche criteria at 2012 which can be provide to niche targeting AI 1018 for matching at 2014. In the event of a match the one or more niche criteria can be applied in niche targeting of one or more profiles at 2020. In the even of no match niche targeting AI 1018 can trigger behavioral analytics engine 1018a and AI matching algorithm 1008 to identify matching profile dynamically at 2016 and/or refine the AI matching techniques at 2018. In embodiments, matched profiles can be provided to API provisioning system 1020, described further hereinafter.

API Provisioning System 1020 can be configured to offer external access to the enriched profiles, search engine, and niche targeting capabilities enabling seamless integration with third-party applications and client systems. In embodiments, API provisioning system 1020 can include one or more sub-modules such as, but not limited to an integration layer module 1018a, and/or an access control module 1018b.

Referring now to a method of using system 1000. A user can access system 1000 through one or more interfaces, such as a web interface by logging into a user-friendly dashboard via a web browser. In embodiments, system 1000 provides intuitive tools for searches, filtering, and data retrieval, as described above. For automated workflows, integrate API Provisioning System 1020 is integrated into existing systems like CRMs, sales automation platforms, or HR systems.

The user can define search parameters and utilize Real-Time Search Engine 1016 to define specific data needs, such as: Job titles and industry (e.g., “VP of Marketing in Real Estate”), Geographical locations (e.g., “Companies operating in the US”), and/or Behavioral attributes. Additionally, the user can refine targeting by entering niche-specific prompts into the AI-Powered Niche Targeting System, such as: “Angel investors interested in Web3 technologies”, “Retail companies with eCommerce capabilities”, “Retailers offering online payment solutions.”, “Profiles with a history of frequent job changes.”. In embodiments, niche targeting AI 1018 can dynamically refine the search, using behavioral analytics to identify patterns or attributes that meet the criteria.

In embodiments, data is retrieved from system 1000, as one or more enriched profiles. In embodiments, the user can view enriched profiles, including real-time updates of contact details such as emails, phone numbers, and job titles. Additionally, the user can export data by downloading the data in CSV, Excel, PDF, or JSON formats for direct use in sales outreach, HR screening, or business analysis.

In embodiments, the data can be verified and enriched, prior to export, or exposure to the user. In embodiments, The Verification Layers such as catch-all verification 1012 and/or phone number verification 1014 can ensure accuracy before the data is exported. For example, email addresses are validated in real time with a 98% success rate, and phone numbers are verified, enriched, and geo-prioritized for regional relevance. In embodiments, the verified data can be utilized for targeted email or phone campaigns, with confidence in the accuracy and relevance of the information. Additionally, the verified data can be used to deploy segmented marketing strategies based on real-time, niche-specific profiles.

System 100 can be integrated with CRM systems like Salesforce or HubSpot using APIs, in order to automate lead generation, contact updates, and data retrieval workflows with minimal manual intervention. System 1000 can provide monitoring and optimization by analyzing campaign performance and user interactions to provide feedback. System 1000 learns from this feedback, refining future targeting and data recommendations via Machine Learning Models.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

The server and the computer of the present invention may each include computing systems. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In some embodiments, the computing systems may execute any suitable operating system such as IBM's zSeries/Operating System (z/OS), MS-DOS, PC-DOS, Mac-OS, Windows, Unix, OpenVMS, an operating system based on Linux, or any other appropriate operating system, including future operating systems. In some embodiments, the computing systems may be a web server running web server applications such as Apache, Microsoft's Internet Information Server™, and the like.

In particular embodiments, the computing systems include a processor, a memory, a user interface and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface may include hardware, software, or both providing one or more interfaces for communication between a person and the computer systems. As an example, and not by way of limitation, a user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface.

The communication interface includes hardware, software, or both providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example, and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

It should be understood, of course, that the foregoing relates to exemplary embodiments of the subject disclosure and that modifications may be made without departing from the spirit and scope of the subject disclosure as set forth in the following claims.

What is claimed is:

1. A real-time artificial intelligence system for business-to-business contact information management, comprising:
a data sourcing module configured to query a plurality of heterogeneous data sources and collect raw contact data, the data sourcing module comprising: a data crawling framework configured to extract data from online sources;
a contributor network integration module configured to receive data from contributor networks; and
a plurality of partnership API connectors configured to interface with partner data sources;
a data compliance module configured to perform compliance checks on the raw contact data against regulatory requirements including GDPR, CCPA, and PDPL regulations, wherein the data compliance module is configured to block non-compliant data and allow compliant data to proceed for further processing;
an artificial intelligence matching algorithm module configured to align the compliant data with data enrichment requirements, the artificial intelligence matching algorithm module comprising:
a data footprint tracking module configured to track digital footprints of sourced data; and
an email intelligence generation module configured to generate email intelligence;
a data enrichment system configured to create unified enriched profiles by combining multiple data points from the artificial intelligence matching algorithm module, the data enrichment system comprising:
a unified profile module configured to consolidate data points into unified profiles; and
a dynamic update mechanism configured to continuously monitor for new data and automatically update the unified enriched profiles in real-time when new data becomes available;
a catch-all verification system configured to verify email addresses in the unified enriched profiles, the catch-all verification system comprising:
an email verification module configured to perform syntax validation, domain verification, and mail server response analysis; and
a multilayered analysis module configured to perform additional verification;
a phone number verification system configured to verify and enrich phone numbers in the unified enriched profiles, the phone number verification system comprising:
a caller ID matching module configured to perform initial phone number validation;
a geolocation engine configured to prioritize regionally relevant contacts; and a cloud communication AI-validation module configured to perform final phone number verification;
a real-time search engine configured to provide live data retrieval from the unified enriched profiles, the real-time search engine comprising:
a cloud infrastructure configured to ensure scalability; and
a user interface dashboard configured to provide user interaction;
a niche targeting artificial intelligence module configured to dynamically refine targeting based on user-defined criteria, the niche targeting artificial intelligence module comprising:
a behavioral analytics engine configured to identify nuanced behavioral patterns; and
a niche-specific targeting engine configured to apply niche-specific targeting based on operational cues and user-defined prompts;
one or more machine learning models configured to continuously improve system components through feedback learning, the one or more machine learning models comprising:
at least one continuous learning model configured to learn from user interactions and data patterns; and
at least one improvement pattern model configured to identify improvement patterns; and
an API provisioning system configured to provide external access to the unified enriched profiles and enable integration with third-party applications, the API provisioning system comprising:
an integration layer module configured to facilitate third-party integration; and
an access control module configured to control access to system resources.

2. The system of claim 1, wherein the data sourcing module is configured to iteratively query the plurality of heterogeneous data sources until requested data is found.

3. The system of claim 1, wherein the catch-all verification system is configured to flag profiles with failed email verification for reprocessing by the data sourcing module and the artificial intelligence matching algorithm module to identify alternative email addresses.

4. The system of claim 1, wherein the phone number verification system is configured to retry verification or employ alternative verification techniques when a phone number fails verification at any verification step.

5. The system of claim 1, wherein the real-time search engine is configured to apply filters to search queries and check niche criteria, and wherein the real-time search engine is configured to refine and resubmit searches when no match is found.

6. The system of claim 1, wherein the niche targeting artificial intelligence module is configured to trigger the behavioral analytics engine and the artificial intelligence matching algorithm module to dynamically identify matching profiles when user-defined niche criteria do not initially match existing profiles.

7. The system of claim 1, wherein the one or more machine learning models are configured to:
analyze user feedback to determine whether the user feedback is positive or negative;
update the one or more machine learning models when the user feedback is positive; and
adjust algorithms when the user feedback is negative.

8. The system of claim 1, wherein the data compliance module is configured to control the API provisioning system, validate information from the real-time search engine, and monitor the data sourcing module.

9. The system of claim 1, wherein the dynamic update mechanism is configured to: continuously monitor for new data availability;
trigger the data sourcing module to perform a dynamic update when new data is found; and
enrich profiles with the new data.

10. The system of claim 1, wherein the email verification module is configured to store verified email data and reprocess failed email data by querying additional data sources.

11. The system of claim 1, wherein the phone number verification system is configured to store phone numbers as verified phone numbers only after the phone numbers pass caller IO matching verification, geolocation prioritization, and cloud communication AI verification.

12. The system of claim 1, wherein the niche targeting artificial intelligence module is configured to identify:
angel investors not explicitly listing their role but engaging in investment-related activities;
eCommerce retailers by analyzing operational cues including add-to-cart features; and
profiles based on user-defined prompts that differ from broader category classifications.

13. The system of claim 1, wherein the system is configured to provide email accuracy of at least 98% and phone number accuracy of at least 95%.

14. The system of claim 1, wherein the system provides access to over one billion contacts.

15. The system of claim 1, wherein the unified enriched profiles include real-time updates of contact details including emails, phone numbers, and job titles.

16. The system of claim 1, wherein the API provisioning system is configured to integrate with customer relationship management systems including Salesforce and HubSpot to automate lead generation, contact updates, and data retrieval workflows.

17. The system of claim 1, wherein the behavioral analytics engine is configured to refine targeting based on industry-specific attributes and user-defined criteria.

18. The system of claim 1, wherein the data sourcing module is configured to continue querying alternative data sources until data is located or all available sources are exhausted.

19. The system of claim 1, wherein the system is configured to export data in formats selected from the group consisting of csv, Excel, PDF, and JSON.

20. The system of claim 1, wherein the niche targeting artificial intelligence module is configured to dynamically refine targeting in real-time based on user-defined prompts rather than relying on static lists.
